(19) United States
(12) Patent Application Publication
(10) Pub. No.: US 2018/0318819 A1
(43) Pub. Date: Nov. 8, 2018

(54) NICKEL-BASED CATALYTIC COMPOSITION IN THE PRESENCE OF A SPECIFIC ACTIVATOR AND USE THEREOF IN A OLEFIN OLIGOMERSATION METHOD

(71) Applicant: IFP Energies nouvelles, Rueil-Malmaison (FR)

(72) Inventors: Pierre-Alain BREUIL, Lyon (FR); Emmanuel PELLIER, Tupin Semons (FR)

(73) Assignee: IFP Energies nouvelles, Rueil-Malmaison (FR)

(21) Appl. No.: 15/772,374

(22) PCT Filed: Oct. 20, 2016
(86) PCT No.: PCT/EP2016/075288

§ 371 Date: Apr. 30, 2018

(30) Foreign Application Priority Data
Oct. 30, 2015 (FR) 1560432

(51) Int. Cl. B01J 31/40; C07C 2/32

(52) U.S. Cl. CPC B01J 31/403; B01J 31/2247; C07C 2/32

(57) ABSTRACT

The invention describes a novel nickel-based composition. The invention also concerns the use of said composition as a catalytic composition in an olefin oligomerization process.

The present invention relates to a novel nickel-based composition. The invention also relates to the use of said composition as a catalyst for chemical transformation reactions.

### PRIOR ART

The preparation of catalytic compositions based on transition metals for application thereof in various fields of chemistry is known, in particular in the field of catalytic transformations such as hydroformylation, hydrogenation, cross-coupling, olefin oligomerization, etc.

The preparation of catalytic compositions of this type depends on the choice of metal, the activating agent and on appropriate ligands.

The document EP 2 220 099 B1 describes a system of coordination complexes comprising multidentate ligands with formula: R₁—SO₂—NH——P(XR₂)₂; or R₁—SO₂—N=PH(XR₂)₂, or R₁—SO(OH)=NP(XR₂)₂, in which X is independently O, S, NH, or a bond; in which R₁ and R₂ are independently selected from an alkyl group, which may or may not be substituted, and an aryl group, in which at least one equivalent of ligand is complexed with one equivalent of a metal selected from rhodium, iridium, platinum, palladium and the lanthanides. EP 2 220 099 B1 indicates that the coordination complex system may be used as a catalyst for hydroformylation, hydrogenation, polymerisation, isomerisation etc. That document does not describe a catalytic system based on nickel with an oxidation number of(+II) and does not mention the use of the specific activating agent in accordance with the invention, in particular in a process for the oligomerization of olefins.

In its research, the Applicant has developed a novel composition comprising a nickel precursor, a sulfonamido-phosphine ligand or a mixture of sulfonamides with phosphine halides, optionally in the presence of a Lewis base, and at least one specific activating agent. Surprisingly, it has been shown that such compositions have interesting catalytic properties. In particular, these compositions have a good catalytic activity and a good selectivity in the oligomerization of olefins, in particular in the dimerization of ethylene to form 1-butene.

One aim of the invention is to provide a novel composition based on nickel(+II). Another aim of the invention is to propose a novel catalytic system comprising said composition for chemical transformation reactions, in particular for the oligomerization of olefins.

### DETAILED DESCRIPTION OF THE INVENTION

Composition in Accordance with the Invention

The catalytic composition of the invention comprises:
at least one precursor of nickel with an oxidation number of(+II),
at least one activating agent with formula [R₃₋ₘAlXₘ]ₙ, the group X being an alkoxy group —OR or a halogen, the groups R being mutually identical or different and selected from alkyl or aromatic groups, n having a value of 1 to 2, m having a value of 1 to 3,
and at least one mixture of a phosphine halide YP(AR^{1a}) (A′R^{1b}) and a sulfonamide compound with formula R²SO₂NH₂, optionally in the presence of a Lewis base denoted Z,

in which
A and A′, which may be identical or different, are independently O, S, NR³ or a single bond between the phosphorus atom and a carbon atom,
the group R³ is either a hydrogen atom or an alkyl group, which may or may not be cyclic, which may or may not be substituted and which may or may not contain heteroelements, or an aromatic group, which may or may not be substituted and which may or may not contain heteroelements,
the groups R¹, represented by R^{1a} and R^{1b}, with R^{1a} and R^{1b} being mutually identical or different and which may or may not be bonded together, are selected from alkyl groups which may or may not be cyclic, which may or may not be substituted and which may or may not contain heteroelements, and from aromatic groups, which may or may not be substituted and which may or may not contain heteroelements,
the group R² is selected from alkyl groups, which may or may not be cyclic, which may or may not be substituted and which may or may not contain heteroelements, and aromatic groups, which may or may not be substituted and which may or may not contain heteroelements,
the group Y being a halogen, preferably chlorine.

In accordance with a variation of the invention, the catalytic composition in accordance with the invention comprises:
at least one precursor of nickel with an oxidation number of(+II),
at least one activating agent with formula [R₃₋ₘAlXₘ]ₙ, the group X being an alkoxy group —OR or a halogen, the groups R being mutually identical or different and selected from alkyl or aromatic groups, n having a value of 1 to 2, m having a value of 1 to 3,
and at least one ligand having tautomeric forms with formula 1a), 1b) and/or 1c):

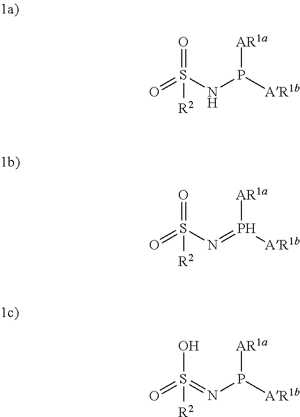

optionally in the presence of a Lewis base denoted Z,

in which composition
A and A′, which may be identical or different, are independently O, S, NR³ or a single bond between the phosphorus atom and a carbon atom,
the group R³ is either a hydrogen atom or an alkyl group, which may or may not be cyclic, which may or may not be substituted and which may or may not contain heteroelements, or an aromatic group, which may or may not be substituted and which may or may not contain heteroelements,
the groups R¹, represented in the formulae 1a), 1b) and 1c) by R^{1a} and R^{1b}, with R^{1a} and R^{1b} being mutually identical or different and which may or may not be bonded together, are selected from alkyl groups which may or may not be cyclic, which may or may not be substituted and which may or may not contain heteroelements, and from aromatic groups, which may or may not be substituted and which may or may not contain heteroelements,
the group R² is selected from alkyl groups, which may or may not be cyclic, which may or may not be substituted and which may or may not contain heteroelements, and aromatic groups, which may or may not be substituted and which may or may not contain heteroelements.

In the context of the present invention, the term “alkyl” is intended to mean a linear or branched hydrocarbon chain containing 1 to 15 carbon atoms, preferably 1 to 10. Examples of preferred alkyl groups are advantageously selected from methyl, ethyl, propyl, isopropyl, butyl, isobutyl and tert-butyl groups. These alkyl groups may be substituted with heteroelements or groups containing heteroelements, such as a halogen or an alkoxy group. The term “alkoxy” substituent means an alkyl-O— group in which the term “alkyl” has the meaning given above. Preferred examples of alkoxy substituents are methoxy or ethoxy groups.

The term “cyclic alkyl” means a monocyclic hydrocarbon group containing more than 3 carbon atoms, preferably 4 to 24, more preferably 5 to 12, preferably a cyclopentyl, cyclohexyl, cyclooctyl or cyclododecyl group, or a polycyclic(bi- or tricyclic) group containing more than 3 carbon atoms, preferably 4 to 18, such as adamantyl or norbornyl groups, for example.

The term “aromatic” means a mono- or polycyclic aromatic group, preferably mono- or bicyclic, containing 5 to 20 carbon atoms. When the group is polycyclic, i.e. it comprises more than one cyclic ring, the cyclic rings may advantageously be condensed in pairs or connected in pairs via σ bonds. The aromatic group in accordance with the invention may contain heteroelements such as nitrogen, oxygen or sulfur.

When the alkyl or aryl groups are substituted with heteroelements or contain heteroelements, these heteroelements are preferably selected from oxygen, nitrogen, sulfur and phosphorus.

The term “ligand” as used in the present invention is used indiscriminately to mean one or more of the tautomeric forms with formula 1a), 1b) and /or 1c) used to form the composition of the invention.

Advantageously in accordance with the invention, the groups R¹, i.e. R^{1a} and R^{1b}, which may be mutually identical or different and which may or may not be bonded together, are independently selected from alkyl groups, which may or may not be cyclic, which may or may not be substituted and which may or may not contain heteroelements, preferably from alkyl groups containing 1 to 15 carbon atoms, preferably 1 to 10 carbon atoms and which may or may not contain heteroelements; and from aromatic groups, which may or may not be substituted and which may or may not contain heteroelements, preferably aromatic groups containing 5 to 20 carbon atoms, which may or may not be substituted, and which may or may not contain heteroelements.

The two groups R¹ (R^{1a} and R^{1b}) may be mutually identical or different. These two groups R^{1a} and R^{1b} may also be bonded together. In such a case, the two groups R^{1a} and R^{1b} may correspond to groups such as bis-phenyl or bis-naphthyl.

Preferably, the groups R¹, i.e. R^{1a} and R^{1b} which may be identical or different, which may or may not be bonded together, are independently selected from methyl, trifluoromethyl, ethyl, n-propyl, i-propyl, n-butyl, i-butyl, t-butyl, pentyl, cyclohexyl and adamantyl groups, which may or may not be substituted and which may or may not contain heteroelements; and from phenyl, o-tolyl, m-tolyl, p-tolyl, mesityl, 3,5-dimethylphenyl, 4-n-butylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di-tert-butyl-4-methoxyphenyl, 4-chlorophenyl, 3,5-di(trifluoromethyl)phenyl, benzyl, naphthyl, bisnaphthyl, pyridyl, bisphenyl, furanyl and thiophenyl groups, which may or may not be substituted and which may or may not contain heteroelements. Preferably, the groups R¹, i.e. R^{1a} and R^{1b}, which may be identical or different, which may or may not be bonded together, are independently selected from phenyl, o-tolyl, m-tolyl, p-tolyl, mesityl, 3,5-dimethylphenyl, 4-n-butylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di-tert-butyl-4-methoxyphenyl, 4-chlorophenyl, 3,5-di(trifluoromethyl)phenyl, benzyl, naphthyl, bisnaphthyl, pyridyl, bisphenyl, furanyl and thiophenyl groups, which may or may not be substituted and which may or may not contain heteroelements. Preferably, the groups R¹, i.e. R^{1a} and R^{1b}, are selected from phenyl or o-tolyl groups.

Advantageously, in accordance with the invention, the groups R² are selected from alkyl groups, which may or may not be cyclic, which may or may not be substituted and which may or may not contain heteroelements, preferably from alkyl groups containing 1 to 15 carbon atoms, preferably 1 to 10 carbon atoms and which may or may not contain heteroelements; and from aromatic groups, which may or may not be substituted and which may or may not contain heteroelements, preferably aromatic groups containing 5 to 20 carbon atoms, which may or may not be substituted and which may or may not contain heteroelements.

Preferably, the groups R² are selected from methyl, trifluoromethyl, ethyl, n-propyl, i-propyl, n-butyl, i-butyl, t-butyl, pentyl, cyclohexyl and adamantyl groups, which may or may not be substituted and which may or may not contain heteroelements; and from phenyl, o-tolyl, m-tolyl, p-tolyl, mesityl, 3,5-dimethylphenyl, 4-n-butylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-ditert-butyl-4-methoxyphenyl, 4-chlorophenyl, 3,5-bis(trifluoromethyl)phenyl, benzyl, naphthyl, bisnaphthyl, pyridyl, bisphenyl, furanyl and thiophenyl groups, which may or may not be substituted and which may or may not contain heteroelements. Preferably, the group R² is trifluoromethyl or 4-n-butylphenyl.

Advantageously in accordance with the invention, the group R³ is either a hydrogen atom or an alkyl group, which may or may not be cyclic, which may or may not be substituted and which may or may not contain heteroelements, preferably an alkyl group containing 1 to 15 carbon atoms, preferably 1 to 10 carbon atoms and which may or may not heteroelements; or an aromatic group, which may or may not substituted and which may or may not contain heteroelements, preferably an aromatic group containing 5 to 20 carbon atoms, which may or may not substituted, which may or may not contain heteroelements. Preferably, the group R³ is either a hydrogen atom or an alkyl group in accordance with the invention.

The composition in accordance with the invention comprises the mixture of the phosphine halide YP(AR^{1a}) (A′R^{1b}) and sulfonamide compound with formula R²SO₂NH₂, in the presence of a Lewis base denoted Z.

By means of a condensation reaction of the phosphine halide YP(AR^{1a}) (A′R^{1b}) and the sulfonamide compound with formula R²SO₂NH₂ in the presence of a Lewis base denoted Z, said mixture is capable of forming the ligand having the tautomeric forms with formula 1a), 1b) and/or 1c).

The composition in accordance with the invention comprises an activating agent with formula [R₃₋ₘAlXₘ]ₙ, the group X being an alkoxy group —OR or a halogen, the groups R being mutually identical or different and selected from alkyl or aromatic groups, n having a value of 1 to 2, m having a value of 1 to 3. Preferably, the indices m and n of the activating agent [R₃₋ₘAlXₘ]ₙ are whole numbers. Preferably, m is in the range 1 to 2.

Preferably, the activating agent is diethylaluminium chloride(Et₂AlCl) and/or diethylaluminium ethoxide(Et₂AlOEt). The activating agents with formula [R₃₋ₘAlXₘ]ₙ may be generated from the association of trialkylaluminium AlR₃ and at least one alcohol of the type ROH.

The composition in accordance with the invention may also comprise a Lewis base denoted Z. This Lewis base Z in accordance with the invention may be an ether O(R⁴)₂ or a tertiary amine N(R⁴)₃, the groups R⁴, which are mutually identical or different, which may or may not be bonded together, being independently selected from alkyl groups which may or may not be cyclic, which may or may not be substituted and which may or may not contain heteroelements, preferably from alkyl groups containing 1 to 15 carbon atoms, preferably 1 to 10 carbon atoms and which may or may not contain heteroelements; and from aromatic groups, which may or may not be substituted and which may or may not contain heteroelements, preferably aromatic groups containing 5 to 20 carbon atoms, which may or may not be substituted, and which may or may not contain heteroelements. The ether is preferably selected from diethylether, methyl tert-butyl ether, di-tert-butyl ether, tetrahydrofuran or dioxane, used alone or as a mixture. The tertiary amine is preferably selected from triethylamine, pyridine, 1,4-diazabicyclo [2.2.2]octane, 1,8-diazabicyclo[5.4.0]undec-7-ene or N-methylmorpholine, used alone or as a mixture.

When the composition in accordance with the invention comprises the Lewis base, together with the ligand with the tautomeric forms 1a), 1b) and/or 1c), this may form an adduct 1d) having the formula corresponding to 1a).Z, 1b).Z and/or 1c).Z.

In a variation, in the case in which the composition in accordance with the invention comprises the Lewis base Z, in addition to the tautomeric forms 1a), 1b) and/or 1c), the composition may comprise at least one adduct 1d) formed between said tautomeric forms and the Lewis base Z and having the formula la).Z, 1b).Z and/or 1c).Z. In such a case, these adducts may coexist with the tautomeric forms 1a), 1b) and/or 1c).

In the case in which the composition in accordance with the invention comprises the mixture of a phosphine halide YP(AR^{1a}) (A′R^{1b}) and a sulfonamide compound with formula R²SO₂NH₂, the adduct 1d) is capable of being formed in the presence of the Lewis base Z.

Preferably, the adduct 1d) is the adduct formed with the tertiary amine, with formula 1a).N(R⁴)₃, R⁴ complying with the specifications of the invention. More preferably, the adduct 1d) has the formula 1a).NEt₃.

The compositions in accordance with the invention may or may not be in the presence of a solvent. It is possible to use a solvent selected from organic solvents, in particular from ethers, alcohols, chlorine-containing solvents and saturated, unsaturated, aromatic or non-aromatic, cyclic or non-cyclic hydrocarbons. Preferably, the solvent is selected from hexane, cyclohexane, methylcyclohexane, heptane, butane or isobutane, monoolefins or diolefins preferably containing 4 to 20 carbon atoms, cycloocta-1,5-diene, benzene, toluene, ortho-xylene, mesitylene, ethylbenzene, dichloromethane, chlorobenzene, methanol or ethanol, either pure or as a mixture, and ionic liquids. In the case in which the solvent is an ionic liquid, it is advantageously selected from the ionic liquids described in patents U.S. Pat. No. 6,951,831 B2 and FR 2 895 406 B1.

Advantageously, in accordance with the invention, the nickel precursor with oxidation number(+II) is selected from nickel(II) chloride, nickel(II) (dimethoxyethane) chloride, nickel(II) bromide, nickel(II) (dimethoxyethane) bromide, nickel(II) fluoride, nickel(II) iodide, nickel(II) sulfate, nickel(II) carbonate, nickel(II) dimethylglyoxime, nickel(II) hydroxide, nickel(II) hydroxyacetate, nickel(II) oxalate, nickel(II) carboxylates such as for example nickel(II) 2-ethylhexanoate, nickel(II) acetate, nickel(II) trifluoroacetate, nickel(II) triflate, nickel(II) acetylacetonate, nickel(II) hexafluoroacetylacetonate, nickel(II) phenates, allylnickel(II) chloride, allylnickel(II) bromide, methallylnickel(II) chloride dimer, allylnickel(II) hexafluorophosphate, methallylnickel(II) hexafluorophosphate, biscyclopentadienyl nickel(II), bisallyl nickel(II) and bismethallyl nickel(II); in their hydrated or non-hydrated form, used alone or as a mixture.

In accordance with the invention, the molar ratio between the ligand with formula 1a), 1b), 1c) and/or 1d) and the nickel precursor is preferably in the range 0.05 to 10, preferably in the range 0.5 to 3.

In accordance with the invention, the molar ratio between the activating agent and the nickel precursor is preferably in the range 1 to 500, preferably in the range 1 to 100, preferably in the range 1 to 30, preferably in the range 2 to 15.

A non-exhaustive list of ligands which may be suitable for the preparation of the compositions of the invention is represented below. The ligands here are represented in their limiting forms 1a) and 1b).

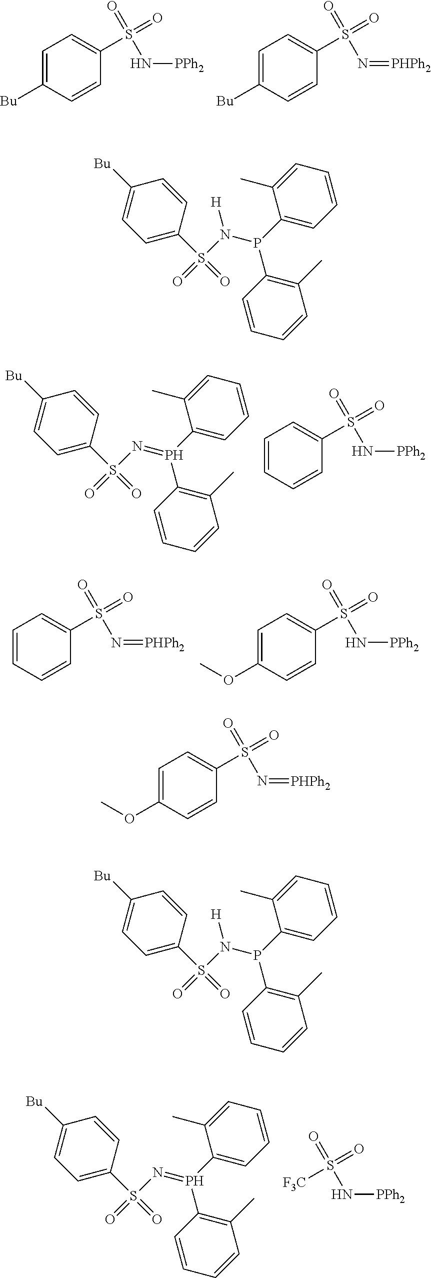

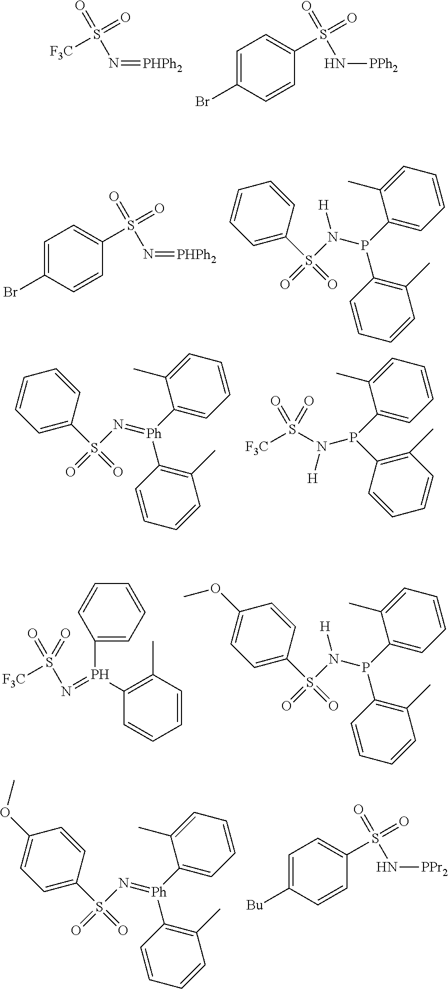

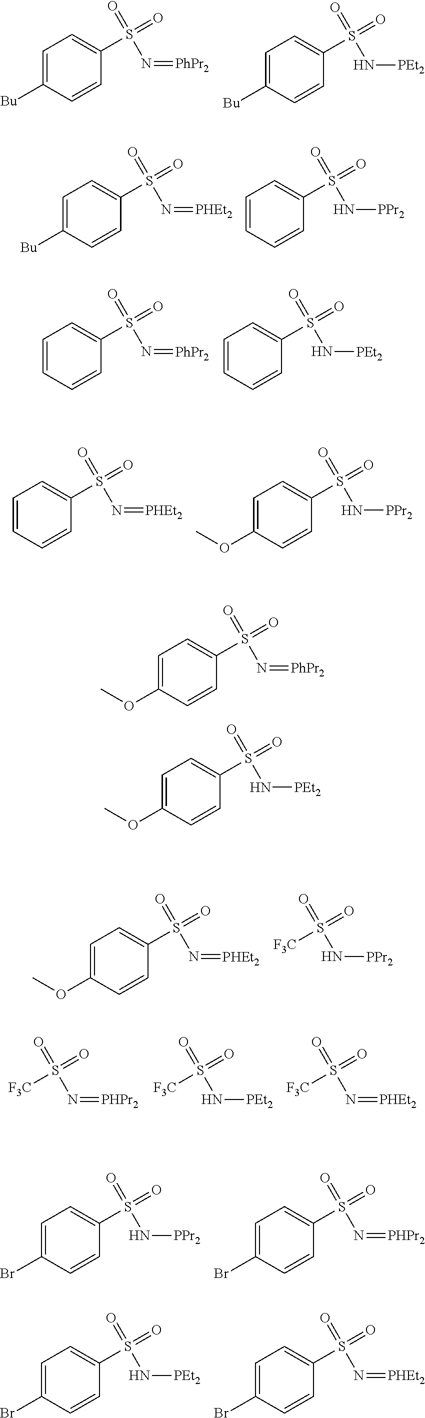

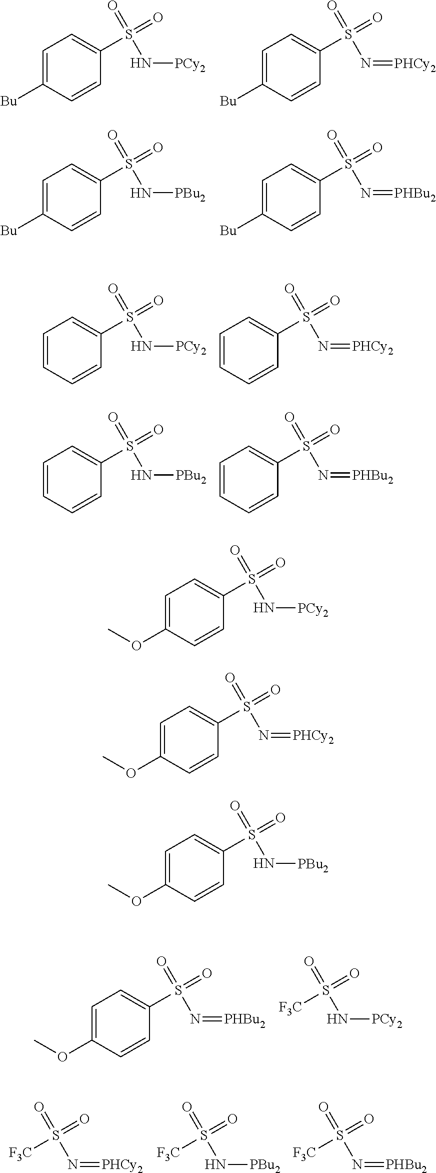

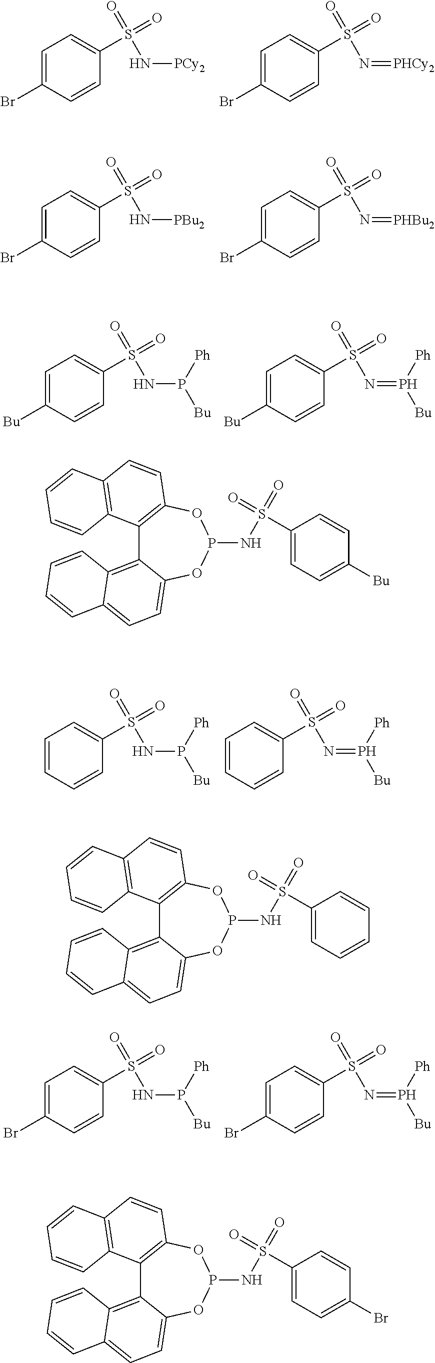

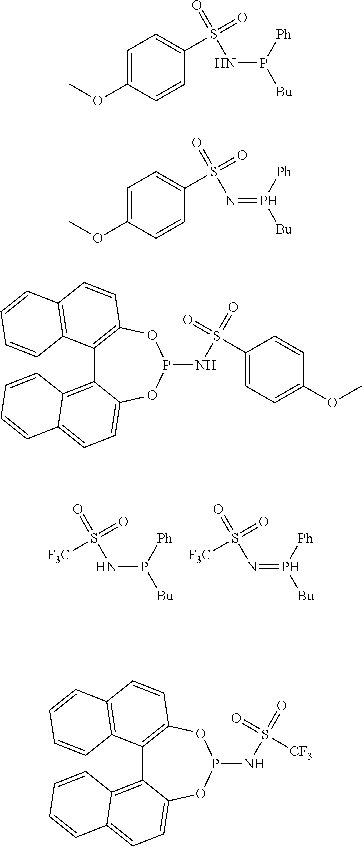

Preparation of the Composition in Accordance with the Invention

The composition in accordance with the invention may be obtained via a mixture between the phosphine halide YP(AR^{1a}) (A′R^{1b}) and the sulfonamide compound with formula R²SO₂NH₂, in the presence of the Lewis base denoted Z, the nickel precursor with oxidation number(+II), and the activating agent with formula [R₃₋ₘAlXₘ]ₙ.

The composition in accordance with the invention may also be obtained via a mixture of the ligand having the tautomeric forms with formula 1a), 1b) and /or 1c), the nickel precursor with oxidation number(+II), and the activating agent with formula [R₃₋ₘAlXₘ]ₙ.

These compositions may also comprise at least the adduct 1d).

Use of the Composition in Accordance with the Invention

The compositions in accordance with the invention may be used as a catalyst in a chemical transformation reaction such as an olefin oligomerization, hydrogenation, hydroformylation, or cross coupling reaction. In particular, these complexes are used in a process for the oligomerization of a feed of olefins advantageously containing 2 to 10 carbon atoms.

Preferably, the oligomerization process is a process for the dimerization of ethylene to 1-butene.

The solvent for the oligomerization process may be selected from organic solvents, preferably from ethers, alcohols, chlorine-containing solvents and saturated, unsaturated, aromatic or non-aromatic, cyclic or non-cyclic hydrocarbons. In particular, said solvent is selected from hexane, cyclohexane, methylcyclohexane, heptane, butane or isobutane, monoolefins or diolefins preferably containing 4 to 20 carbon atoms, benzene, toluene, ortho-xylene, mesitylene, ethylbenzene, dichloromethane, chlorobenzene, methanol and ethanol, pure or as a mixture, and ionic liquids. In the case in which said reaction solvent is an ionic liquid, it is advantageously selected from the ionic liquids described in patents U.S. Pat. No. 6,951,831 B2 and FR 2 895 406 B1.

Oligomerization is defined as the transformation of a monomer unit into a compound or mixture of compounds with general formula CₚH₂ₚ, with 4≤p≤80, preferably with 4≤p≤50, more preferably with 4≤p≤26 and highly preferably with 4≤p≤14.

The olefins used in the oligomerization process are olefins containing 2 to 10 carbon atoms. Preferably, said olefins are selected from ethylene, propylene, n-butenes and n-pentenes, alone or as a mixture, pure or diluted. Preferably, the olefin used in the oligomerization process is ethylene.

In the case in which said olefins are diluted, said olefins are diluted with one or more alkane(s) such as those found in the “cuts” obtained from oil refining processes such as catalytic cracking or steam cracking.

Said olefins of the feed may be obtained from non-fossil sources such as biomass. As an example, the olefins used in the oligomerization process in accordance with the invention may be produced from alcohols, in particular by dehydration of alcohols.

The concentration of nickel in the reactor is advantageously in the range 1×10⁻⁸ to 1 mol/L, and preferably in the range 1×10⁻⁶ to 1×10⁻² mol/L.

The oligomerization process is advantageously operated at a total pressure in the range between atmospheric pressure and 20 MPa, preferably in the range 0.1 to 8 MPa, and at a temperature in the range −40° C. to +250° C., preferably in the range −20° C. to 150° C.

The heat generated by the reaction may be eliminated using any means known to the skilled person.

In a particular embodiment, the oligomerization process in accordance with the invention comprises:
a) a step for condensation of a phosphine halide YP(AR^{1a}) (A′R^{1b}) and a sulfonamide compound with formula R²SO₂NH₂, optionally in the presence of the Lewis base in accordance with the invention,
b) optionally, a step for separation of the reaction product obtained from step a) in order to obtain the ligand having the tautomeric forms 1a), 1b) and/or 1c), and/or optionally the adduct 1d),
c) a step for bringing the feed into contact with the reaction product obtained from step a) or the ligand having the tautomeric forms 1a), 1b) and/or 1c), and/or optionally the adduct 1d) obtained from the separation step b), the nickel precursor and the activating agent.

The term “separation step” means any step for the purification of the reaction product of step a) which can isolate the impurities from the ligand with formula 1a), 1b), 1c) or 1d) or their mixture. The separation step may, for example, be a step for filtration.

Step a) is preferably carried out at a temperature in the range −80° C. to 120° C., advantageously in an organic solvent. The Lewis base may be used in excess with respect to the reagents for the condensation reaction of step a), for example between 1.1 and 3 equivalents per equivalent of reagent. The phosphine halide YP(AR^{1a}) (A′R^{1b}) and sulfonamide compound reagents with formula R²SO₂NH₂ are advantageously reacted in stoichiometric quantities. The phosphine halide YP(AR^{1a}) (A′R^{1b}) may be used in a molar ratio with respect to the sulfonamide compound with formula R²SO₂NH₂ which is in the range 0.2 to 1.

The oligomerization process may be carried out in a closed system, in a semi-open system or continuously, with one or more reaction stages. Vigorous stirring is advantageously carried out in order to ensure good contact between the reagent or reagents and the catalytic system.

Advantageously, the oligomerization process is carried out by introducing a mixture of the nickel precursor and the ligand and/or optional adduct 1d) obtained from the separation step b), or a mixture of nickel precursor and reaction product obtained from step a) on the one hand, and on the other hand, introducing the activating agent into a reactor, in the presence of the feed, provided with the usual stirring, heating and cooling devices.

The oligomerization process may be carried out discontinuously. In this case, a selected volume of the solution comprising the composition in accordance with the invention is introduced into a reactor provided with the usual stirring, heating and cooling devices.

The oligomerization process may also be carried out in a continuous manner. In this case, the solutions comprising the elements of the composition of the invention are injected at the same time as the olefin into a reactor stirred using conventional mechanical means or by external recirculation, maintaining the desired temperature.

The catalytic composition is destroyed by any usual means known to the skilled person, then the reaction products as well as the solvent are separated, for example by distillation. The olefin which has not been transformed may be recycled to the reactor.

The process in accordance with the invention may be carried out in a reactor with one or more reaction stages in series, the olefinic feed and/or the catalytic composition, having been pre-conditioned, being introduced continuously, either into the first stage or into the first and any other of the stages. At the reactor outlet, the catalytic composition may be deactivated, for example by injecting ammonia and/or an aqueous solution of sodium hydroxide and/or an aqueous solution of sulfuric acid. The unconverted olefins and any alkanes which might be present in the feed are then separated from the oligomers by distillation.

The products of the present process may find an application, for example, as fuel components for automobiles, as feeds in a hydroformylation process for the synthesis of aldehydes and alcohols, as components for the chemicals, pharmaceuticals or perfumery industry and/or as feeds in a metathesis process for the synthesis of propylene, for example.

The following examples illustrate the invention without limiting its scope.

### EXAMPLE 1

### Synthesis of Isolated Ligands

Synthesis of Ligand SP1

Trifluoromethane sulfonamide(2.4 g, 16 mmol, 1 eq.) and triethylamine Z(4.2 g, 40 mmol, 2.6 eq., 6 mL) were dissolved in 30 mL of tetrahydrofuran(THF). In a second Schlenk tube, di(o-tolyl)chlorophosphine was dissolved with 10 mL of THF. The solution of chlorophosphine(4 g, 16 mmol, 1 eq.) in 10 mL of THF was added dropwise to the solution of sulfonamide in order to produce a white precipitate. After 20 min, the mixture was filtered and the solid was rinsed twice with 10 mL of THF. The liquid phase was evaporated under vacuum in order to obtain a colourless oil. 20 mL of diethylether was added to said oil in order to precipitate a white powder. The powder was rinsed three times with 5 mL of diethylether. After vacuum drying, 2.52 g of powder was obtained, i.e. a yield of 68%.

The major product SP1, corresponding to the tautomeric form 1d) =1 a).Z with Z =NEt₃, was characterized by ³¹P{¹H} NMR(C₆D₆), ³¹P NMR(C₆D₆), ¹H NMR(C₆D₆) and ¹³C NMR (C₆D₆) spectroscopy.

³¹P NMR(C₆D₆):

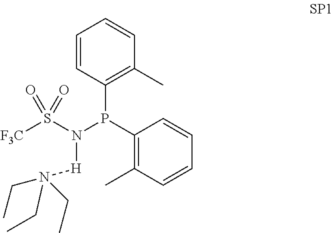

Synthesis of Ligand SP2

Trifluoromethane sulfonamide(2.4 g, 16 mmol, 1 eq.) and triethylamine Z(4.2 g, 40 mmol, 2.6 eq., 6 mL) were dissolved in 30 mL of tetrahydrofuran(THF). In a second Schlenk tube, di(o-tolyl)chlorophosphine was dissolved with 10 mL of THF. The solution of chlorophosphine(4 g, 16 mmol, 1 eq.) in 10 mL of THF was added dropwise to the solution of sulfonamide in order to produce a white precipitate. After 20 min, the mixture was filtered and the solid was rinsed twice with 10 mL of THF. The triethylamine was eliminated under vacuum at 50° C. The precipitated ligand was rinsed three times with 5 mL of diethylether. After vacuum drying, 3.46 g of powder was obtained, i.e. a yield of 60%.

The major product SP2, corresponding to the tautomeric form 1b), was characterized by ³¹1³{¹H} NMR(C₆D₆), ³¹P NMR(C₆D₆), ¹H NMR(C₆D₆) and ¹³C NMR(C₆D₆) spectroscopy.

³¹P NMR(C₆D₆): 15.8.

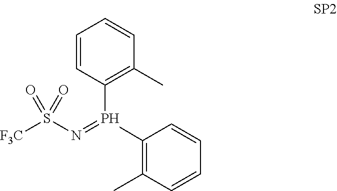

Synthesis of Ligand SP3

The synthesis of ligand SP3, corresponding to the tautomeric form la), was carried out in accordance with the method described in the literature: F. G. Terrade, Eur. J. Inorg. Chem. 2014, 1826-1835.

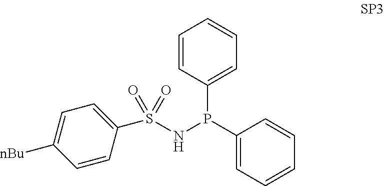

Synthesis of Ligand L(Comparative)

The synthesis of L was carried out in accordance with the method described in the literature: M. S. Balakrishna, J. Organomet. Chem. 1990, 203-216.

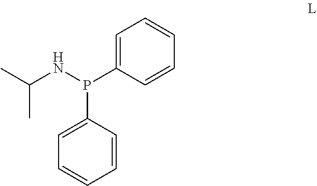 [SMILES: CC(C)NP(c1ccccc1)c1ccccc1]

Carrying Out the ethylene Oligomerization Test

The nature of the components(nickel(II) precursor, ligand and activating agent) as well as their quantities in the various catalytic tests are described in Table 1.

Toluene(94 mL) was introduced into the reactor which had been heated to 40° C. 5 mL of the solution containing the nickel precursor(with nNi=10 μmol) and the ligand was then added. The reaction medium was stirred for 15 minutes under approximately 2 bar of ethylene. The reactor was then degassed. 1 mL of solution containing the activating agent was then added. The reactor was rapidly pressurized(30 bar of ethylene). The reactor was then heated to 40° C.

The test was stopped after 50 g of ethylene had been introduced or after the reaction time indicated in Table 1. The reactor was then depressurized and the gas phase was quantified and qualified by gas phase chromatography. The liquid phase was weighed, neutralized and analysed by gas phase chromatography.

TABLE 1
Evaluation of isolated ligands

| | Catalytic | Ligand | Activating agent | Time | Activity | C4^{a} | C6^{a} | C8^{+a} | 1-C4^{b} |
|---|---|---|---|---|---|---|---|---|---|
| Exx | precursor | (ratio Ligand/Ni) | (ratio Al/Ni) | (minutes) | (10³ g/(g_{Ni} · h)) | (% by wt) | (% by wt) | (% by wt) | (%) |
| 1 | Ni(acac)₂ | SP1 (1) | Et₂AlOEt (2.5) | 30 | 216 | 79.4 | 15.9 | 4.7 | 97.4 |
| 2 | Ni(2-EH)₂ | SP1 (1) | Et₂AlOEt (2.5) | 45 | 168 | 86.4 | 11.8 | 1.8 | 98.9 |
| 3 | Ni(2-EH)₂ | SP1 (2) | Et₂AlOEt (2.5) | 90 | 53 | 89.4 | 9.6 | 1.0 | 99+ |
| 4 | Ni(naph)₂ | SP1 (2) | Et₂AlOEt (2.5) | 30 | 115 | 88.1 | 10.7 | 1.1 | 99+ |
| 5 | Ni(2-EH)₂ | SP1 (2) | Et₂AlCl (15) | 90 | 45 | 96.5 | 3.5 | 0.0 | 94.7 |
| 6 | Ni(2-EH)₂ | SP2 (2) | Et₂AlOEt (2.5) | 30 | 204 | 85.3 | 12.6 | 2.1 | 99+ |
| 7 | Ni(2-EH)₂ | SP3 (2) | Et₂AlOEt (2.5) | 10 | 26 | 84.7 | 9.3 | 6.0 | 99+ |
| 8 | Ni(2-EH)₂ | L (2) | Et₂AlOEt (2.5) | 90 | 0 | — | — | — | — |

Operating conditions: n_{Ni} = 10 μmol, 40° C., 30 bar of ethylene, toluene (100 mL).
^{a}% by wt = percentage by weight with respect to products formed.
^{b}1-C4 (%) = percentage of 1-butene in the C4 cut.
acac = acetylacetonate, naph = naphthenate, 2-EH = 2-ethylhexanoate.
The ratios Ligand/Ni and Al/Ni are expressed as the molar ratios.

The catalytic composition in accordance with the invention(Exxs 1 to 7) exhibited good activity and good selectivity in the dimerization of ethylene into 1-butene compared with the composition of Exx 8, not in accordance with the invention, comprising a ligand L.

Formation of Ligand From A Sulfonamide(s) and a Chlorophosphine(P) and Carrying Out an ethylene Oligomerization Test:

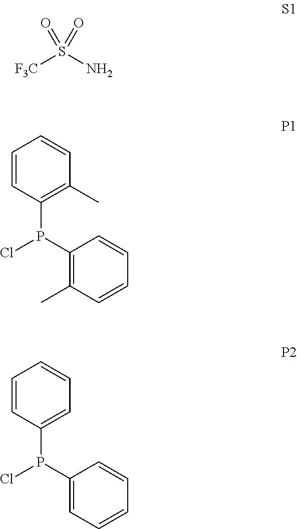 [SMILES: NS(=O)(=O)C(F)(F)F ; Cc1ccccc1P(Cl)c1ccccc1C ; ClP(c1ccccc1)c1ccccc1]

The nature of the components(sulfonamide S, chlorophosphine P, triethylamine Z, catalytic precursor, ligand and activating agent) as well as their respective quantities considered in the various catalytic tests are described in Table 2.

Sulfonamide S(quantity see Table 2) and triethylamine were dissolved in 10 mL of anhydrous toluene. In a second Schlenk tube, the chlorophosphine P(2 mmol) was dissolved with 10 mL of anhydrous toluene, then was added dropwise to the solution of sulfonamide in order to produce a white precipitate. After 10 minutes to 3 hours(the progress of the reaction was monitored by ³¹P NMR), the mixture was filtered. It could contain a mixture of various ligands and/or adducts with formulae 1a), 1b), 1c) and/or 1d). A fraction of 20 μmol of phosphorus equivalent was then removed and added to a solution containing 10 μmol of catalytic nickel precursor Ni(2-EH)₂ the volume of which had been adjusted in order to obtain a total volume of 5 mL.

Toluene(94 mL) was introduced into the reactor which had been heated to 40° C. 5 mL of the solution prepared above, containing the nickel precursor(with nNi=10 μmol) was then added. The reaction medium was stirred for 15 minutes under approximately 2 bar of ethylene. The reactor was then degassed. 1 mL of solution containing the activating agent was then added. The reactor was rapidly pressurized(30 bar of ethylene). The reactor was then heated to 45° C.

The test was stopped after 50 g of ethylene had been introduced or after the reaction time indicated in the Table. The reactor was then depressurized and the gas phase was quantified and qualified by gas phase chromatography. The liquid phase was weighed, neutralized and analysed by gas phase chromatography.

TABLE 2
Evaluation of catalytic compositions from mixtures of sulfonamide S and chlorophosphine P.

| | | Ratio | Ratio | Temp. | Time | Activity | C4^{a} | C6^{a} | C8^{+a} | 1-C4^{b} |
|---|---|---|---|---|---|---|---|---|---|---|
| Exx | Mixture | S/P/Ni | NEt₃/P | (° C.) | (minutes) | (10³ g/(g_{Ni} · h)) | (% by wt) | (% by wt) | (% by wt) | (%) |
| 1 | S1 + P1 | 2/2/1 | 2.6 | 60 | 45 | 40 | 85.7 | 11.8 | 2.5 | 99+ |
| 2 | S1 + P1 | 2/2/1 | 2.0 | 60 | 45 | 61 | 84.4 | 13.7 | 1.9 | 99+ |
| 3 | S1 + P1 | 2/2/1 | 1.1 | 45 | 90 | 23 | 89.3 | 9.6 | 1.1 | 99+ |
| 4 | S1 + P2 | 2.2/2/1 | 1.7 | 60 | 90 | 13 | 98.4 | 1.6 | 0.0 | 96.9 |
| 5 | S1 + P2 | 4/2/1 | 1.4 | 60 | 90 | 20 | 98.1 | 1.8 | 0.1 | 96.0 |

Operating conditions: Catalytic precursor: Ni(2-EH)2, nNi = 10 μmol, Et2AlOEt (Al/Ni = 2.5), 30 bar C2, toluene (100 mL), 90 min. 2-EH = 2-ethylhexanoate.
^{a}% by wt = percentage by weight with respect to products formed.
^{b}1-C4 (%) = percentage of 1-butene in the C4 cut.
the ratio S/P/Ni is expressed as the molar ratio, the ratio NEt₃/P is expressed as the molar ratio.

The catalytic composition in accordance with the invention(Exxs 1 to 5) obtained from the mixture of sulfonamide S and chlorophosphine P in accordance with the invention exhibited good activity and good selectivity for the dimerization of ethylene into 1-butene.

1. A composition comprising:
at least one precursor of nickel with an oxidation number of(+II),
at least one activating agent with formula [R₃₋ₘAlXₘ]ₙ, the group X being an alkoxy group —OR or a halogen, the groups R being mutually identical or different and selected from alkyl or aromatic groups, n having a value of 1 to 2, m having a value of 1 to 3,
and at least one mixture of a phosphine halide YP(AR^{1a}) (A′R^{1b}) and a sulfonamide compound with formula R²SO₂NH₂, optionally in the presence of a Lewis base denoted Z, in which
A and A′, which may be identical or different, are independently O, S, NR³ or a single bond between the phosphorus atom and a carbon atom,
the group R³ is either a hydrogen atom or an alkyl group, which may or may not be cyclic, which may or may not be substituted and which may or may not contain heteroelements, or an aromatic group, which may or may not be substituted and which may or may not contain heteroelements,
the groups R¹, represented by R^{1a} and R^{1b}, with R^{1a} and R^{1b} being mutually identical or different and which may or may not be bonded together, are selected from alkyl groups which may or may not be cyclic, which may or may not be substituted and which may or may not contain heteroelements, and from aromatic groups, which may or may not be substituted and which may or may not contain heteroelements,
the group R² is selected from alkyl groups, which may or may not be cyclic, which may or may not be substituted and which may or may not contain heteroelements, and aromatic groups, which may or may not be substituted and which may or may not contain heteroelements,
the group Y being a halogen.

2. A composition comprising:
at least one precursor of nickel with an oxidation number of(+II),
at least one activating agent with formula [R₃₋ₘAlXₘ]ₙ, the group X being an alkoxy group —OR or a halogen, the groups R being mutually identical or different and selected from alkyl or aromatic groups, n having a value of 1 to 2, m having a value of 1 to 3,
and at least one ligand having tautomeric forms with formula 1a), 1b) and /or 1c) 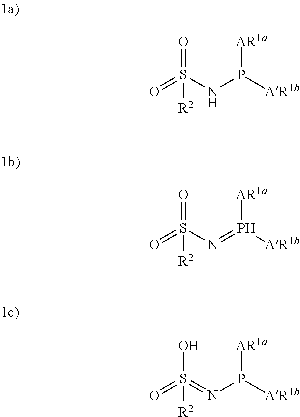
optionally in the presence of a Lewis base denoted Z,
in which composition
A and A′, which may be identical or different, are independently O, S, NR³ or a single bond between the phosphorus atom and a carbon atom,
the group R³ is either a hydrogen atom or an alkyl group, which may or may not be cyclic, which may or may not be substituted and which may or may not contain heteroelements, or an aromatic group, which may or may not be substituted and which may or may not contain heteroelements,
the groups R¹, represented in the formulae 1a), 1b) and 1c) by R^{1a} and R^{1b}, with R^{1a} and R^{1b} being mutually identical or different and which may or may not be bonded together, are selected from alkyl groups which may or may not be cyclic, which may or may not be substituted and which may or may not contain heteroelements, and from aromatic groups, which may or may not be substituted and which may or may not contain heteroelements,
the group R² is selected from alkyl groups, which may or may not be cyclic, which may or may not be substituted and which may or may not contain heteroelements, and aromatic groups, which may or may not be substituted and which may or may not contain heteroelements.

3. The composition as claimed in claim 1, in which the activating agent is diethylaluminium chloride(Et₂AlCl) and/or diethylaluminium ethoxide (Et₂AlOEt).

4. The composition as claimed in claim 1, in which the groups R^{1a} and R^{1b} are independently selected from methyl, trifluoromethyl, ethyl, n-propyl, i-propyl, n-butyl, i-butyl, t-butyl, pentyl, cyclohexyl and adamantyl groups, which may or may not be substituted and which may or may not contain heteroelements; and from phenyl, o-tolyl, m-tolyl, p-tolyl, mesityl, 3,5-dimethylphenyl, 4-n-butylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-di-tert-butyl-4-methoxyphenyl, 4-chlorophenyl, 3,5-di(trifluoromethyl)phenyl, benzyl, naphthyl, bisnaphthyl, pyridyl, bisphenyl, furanyl and thiophenyl groups, which may or may not be substituted and which may or may not contain heteroelements.

5. The composition as claimed in claim 1, in which the groups R² are selected from methyl, trifluoromethyl, ethyl, n-propyl, i-propyl, n-butyl, butyl, t-butyl, pentyl, cyclohexyl and adamantyl groups, which may or may not be substituted and which may or may not contain heteroelements; and from phenyl, o-tolyl, m-tolyl, p-tolyl, mesityl, 3,5-dimethylphenyl, 4-n-butylphenyl, 4-methoxyphenyl, 2-methoxyphenyl, 3-methoxyphenyl, 4-methoxyphenyl, 2-isopropoxyphenyl, 4-methoxy-3,5-dimethylphenyl, 3,5-ditert-butyl-4-methoxyphenyl, 4-chlorophenyl, 3,5-bis(trifluoromethyl)phenyl, benzyl, naphthyl, bisnaphthyl, pyridyl, bisphenyl, furanyl and thiophenyl groups, which may or may not be substituted and which may or may not contain heteroelements.

6. The composition as claimed in claim 1, in which the Lewis base Z is an ether O(R⁴)₂ or a tertiary amine N(R⁴)₃, the groups R⁴, which may be mutually identical or different, which may or may not be bonded together, being independently selected from alkyl groups which may or may not be cyclic, which may or may not be substituted and which may or may not contain heteroelements; and from aromatic groups, which may or may not substituted and which may or may not contain heteroelements.

7. The composition as claimed in claim 6, in which the ether is selected from diethylether, methyl tert-butyl ether, di-tert-butyl ether, tetrahydrofuran or dioxane, used alone or as a mixture, and the tertiary amine is selected from triethylamine, pyridine, 1,4-diazabicyclo[2.2.2]octane, 1,8-diazabicyclo [5.4.0]undec-7-ene or N-methylmorpholine, used alone or as a mixture.

8. The composition as claimed in claim 1, further comprising at least one adduct 1d) formed between the tautomeric forms of the ligand 1a), 1b) and/or 1c) and the Lewis base Z and having the formula 1a).Z, 1b).Z and/or 1c).Z.

9. The composition as claimed in claim 1, in which the nickel precursor with oxidation number(+II) is selected from nickel(II) chloride, nickel(II) (dimethoxyethane) chloride, nickel(II) bromide, nickel(II) (dimethoxyethane) bromide, nickel(II) fluoride, nickel(II) iodide, nickel(II) sulfate, nickel(II) carbonate, nickel(II) dimethylglyoxime, nickel(II) hydroxide, nickel(II) hydroxyacetate, nickel(II) oxalate, nickel(II) carboxylates such as for example nickel(II) 2-ethylhexanoate,nickel(II) acetate, nickel(II) trifluoroacetate, nickel(II) triflate, nickel(II) acetylacetonate, nickel(II) hexafluoroacetylacetonate, nickel(II) phenates, allylnickel(II) chloride, allylnickel(II) bromide, methallylnickel(II) chloride dimer, allylnickel(II) hexafluorophosphate, methallylnickel(II) hexafluorophosphate, biscyclopentadienyl nickel(II), bisallyl nickel(II) and bismethallyl nickel(II); in their hydrated or non-hydrated form, used alone or as a mixture.

10. The composition as claimed in claim 2 in which the molar ratio between the ligand with formula 1a),1b) or 1c) and/or the adduct 1d) and the nickel precursor is in the range 0.05 to 10.

11. The composition as claimed in claim 1, in which the molar ratio between the activating agent and the nickel precursor is in the range 1 to 500.

12. Use of a composition according to claim 1, as a catalyst.

13. A process for oligomerizing an olefinic feed, comprising bringing said feed into contact with a composition according to claim 1.

14. A process for the oligomerization of a feed of olefins as claimed in claim 13, comprising:
a) a step for condensation of a phosphine halide YP(AR^{1a}) (A′R^{1b}) and a sulfonamide compound with formula R²SO₂NH₂, optionally in the presence of the Lewis base,
b) optionally, a step for separation of the reaction product obtained from step a) in order to obtain the ligand having the tautomeric forms 1a), 1b) and/or 1c), and/or optionally the adduct 1d),
c) a step for bringing the feed into contact with the reaction product obtained from step a) or the ligand having the tautomeric forms 1a), 1b) and/or 1c), and/or optionally the adduct 1d) obtained from the separation step b), the nickel precursor and the activating agent.

15. The oligomerization process as claimed in claim 13, carried out by introducing a mixture of the nickel precursor and ligand and/or optional adduct 1d) obtained from the separation step b), or a mixture of nickel precursor and reaction product obtained from step a) on the one hand, and on the other hand, introducing the activating agent into a reactor, in the presence of the feed, provided with the usual stirring, heating and cooling devices.

16. The process as claimed in claim 13, in which the feed comprises olefins containing in the range 2 to 10 carbon atoms.

17. The process as claimed in claim 13, in which the reaction is an ethylene oligomerization reaction.

18. The process as claimed in claim 13, in which the reaction is a reaction for the dimerization of ethylene to 1-butene.
